(19) United States
(12) Patent Application Publication
(10) Pub. No.: US 2021/0196631 A1
(43) Pub. Date: Jul. 1, 2021

(54) CONTAINER SYSTEM AND PHARMACEUTICAL FOAM COMPOSITION COMPRISING BETAMETHASONE

(71) Applicant: Glenmark Pharmaceuticals Limited, Mumbai (IN)

(72) Inventors: Ulhas Dhuppad, Nashik, Maharashtra (IN); Sushrut Kulkarni, Nashik, Maharashtra (IN); Madhusudhan Bommagani, Mumbai (IN); Roopali Amoi Oravkar, Navi Mumbai (IN); Ranjith Kumar Mamidala, Warangal, Telangana (IN); Bhanudas Yadav Shalke, Pure, Maharashtra (IN)

(21) Appl. No.: 17/250,714

(22) PCT Filed: Aug. 27, 2019
(86) PCT No.: PCT/IB2019/057194

§ 371 Date: Feb. 23, 2021

(30) Foreign Application Priority Data
Aug. 28, 2018 (IN) 201821032179

(51) Int. Cl. A61K 9/12; A61K 31/573; A61K 31/593; A61K 47/44; A61K 47/22; A61K 47/06; A61K 47/10; A61F 13/40

(52) U.S. Cl. CPC A61K 9/124; A61K 31/573; A61K 31/593; A61K 47/44; A61M 35/006; A61K 47/06; A61K 47/10; A61K 9/122; A61K 47/22

(57) ABSTRACT

The present invention relates to a pharmaceutical foam composition comprising a corticosteroid and a Vitamin D analogue for topical administration to a patient in need thereof, such as for the treatment of plaque psoriasis. The present invention also relates to a process for preparing the composition and a suitable container system for administration of the composition. Preferably, the invention relates to the topical administration of betamethasone dipropionate and calcipotriene with one or more pharmaceutically acceptable excipients and propellants where the composition is stable in the container system coated with a coating material selected from the group comprising of epoxyphenol resin, modified polyesters, microflex coating or polyacrylates.

### PRIORITY DETAILS

This application claims priority from Indian provisional patent application No. 201821032179 filed on Aug. 28, 2018 which is incorporated here in its entirety.

### FIELD OF THE INVENTION

The present invention relates to a pharmaceutical foam composition comprising a corticosteroid and a Vitamin D analogue for topical administration to a patient in need thereof, such as for the treatment of plaque psoriasis. The present invention also relates to a process for preparing the composition and a suitable container system for administration of the composition. Preferably, the invention relates to the topical administration of betamethasone dipropionate and calcipotriene with one or more pharmaceutically acceptable excipients and propellants where the composition is stable in the container system coated with a coating material selected from the group comprising of epoxyphenol resin, modified polyesters, microflex coating or polyacrylates.

### BACKGROUND OF THE INVENTION

Psoriasis is a chronic, painful, disease which involves skin and nails of a patient. Skin lesions cause pain, stinging and itching, and are mostly symmetrical, demarcated with red papules and plaques and are covered with white or silver scales. Individuals with psoriasis develop chronic, inflammatory arthritis (psoriatic arthritis) that leads to joint deformations and disability and develop nail changes. Individuals with psoriasis are reported to be at increased risk of developing other serious clinical conditions such as cardiovascular and other non-communicable diseases. It is a disfiguring and disabling disease which has a negative impact on the quality of life of a patient. Treatment for Psoriasis includes topical therapy, phototherapy and systemic therapy. Corticosteroids and Vitamin D analogues have been used as topical treatment agents for psoriasis. (Ref: Global report on Psoriasis, World Health Organization, 2016).

Corticosteroids such as betamethasone and vitamin D analogs such as calcipotriene are stable at different pH condition i.e. Betamethasone is stable at a pH of around 4-6 and Calcipotriene is stable at pH greater than 8. This difference in their pH stability conditions, combining the two active agents in a single formulation is challenging. Further a foam formulation comprising a corticosteroids like betamethasone and vitamin D analogs like Calcipotriene is preferred for a patient suffering from plaque psoriasis.

A combination of betamethasone dipropionate and calcipotriene in a topical foam composition is marketed under the trade name Enstilar®. This product is described in U.S. Pat. No. 9,119,781. The patent discloses a topical spray composition comprising a biologically active vitamin D derivative, a corticosteroid, and a propellant which can be dimethyl ether or a mixture of dimethyl ether and one or more C₃₋₅ alkanes. There still however remains a need for a topical foam composition comprises betamethasone and calcipotriene which is stable, provides better absorption of the two active ingredients, is prepared by simple manufacturing process and provides the satisfactory foam characteristics like viscosity, foam consistency and texture.

Further, in such foam formulation comprising two active ingredients and additional pharmaceutical excipients, there is a likelihood of interaction between the drug or excipient and the material of the container. Thus, there is also a pressing need for selection of a suitable coating material for topical foam compositions comprising a combination of betamethasone dipropionate and calcipotriene. The present invention specifically addresses this need and relates to a stable foam composition comprising a betamethasone dipropionate and calcipotriene for topical administration and a container system comprising the same. The container system is coated with coating materials such as epoxyphenol resin, modified polyesters, microflex coating or polyacrylates. The topical compositions of the present invention are stable and compatible with the coating materials.

### SUMMARY OF THE INVENTION

In an embodiment, the present invention relates to a pharmaceutical foam composition comprising about 0.0643% w/w betamethasone dipropionate and about 0.0052% w/w calcipotriene with one or more pharmaceutically acceptable excipients.

In an embodiment, the present invention relates to an ointment based foam composition comprising about 0.0643% w/w betamethasone dipropionate and about 0.0052% w/w calcipotriene for topical administration. The ointment based foam can be administered to a patient in need thereof for the treatment of psoriasis. In one embodiment, the foam breaks down after administration onto the skin and an ointment forms an occlusive layer on the affected area.

In an embodiment, the present invention relates to a stable pharmaceutical foam composition comprising betamethasone dipropionate and calcipotriene with one or more pharmaceutically acceptable excipients for the treatment of psoriasis.

In another embodiment, the present invention relates to a stable foam composition wherein the composition is stable at (i) 40° C. and 75% relative humidity (RH), (ii) 30° C. and 65% RH, or (iii) 25° C. and 60% RH for 1, 2, 3, or 6 months, wherein the related substance impurity C ((5E,7E,22E,24S)-24-cyclopropyl 9,10-secochola-5,7,10(19),22-tetraene-1α,3β 24-triol 5E isomer) content is not more than 3% after storage for the 1, 2, 3, or 6 months.

The compositions of the present invention may include a propellant system. In one embodiment, the propellant system comprises a hydrocarbon propellant or a mixture of hydrocarbon propellants. In one embodiment, the propellant system does not comprises monomethyl ether or dimethyl ether or a any derivative thereof.

In an embodiment, the composition is filled into a container under pressure wherein the pressure ranges from about 65 to about 80 psig.

In another embodiment, the viscosity of the foam composition ranges from about 1 to about 15 poise.

In an embodiment, the foam density of the foam composition ranges from about 0.2 to about 0.5 g/ml.

In an embodiment, the delivery rate of the foam composition dispensed from the container ranges from about 0.5 to about 1 g/sec.

Another embodiment is a pharmaceutical foam composition comprising about 0.0643% w/w betamethasone dipropionate and about 0.0052% w/w calcipotriene with one or more pharmaceutically acceptable excipients, wherein the composition comprises about 40 to about 95% w/w white petrolatum, about 1 to about 10% w/w mineral oil, about 0.001 to about 0.01% w/w alpha tocopherol, about 1 to about 20% w/w isopropyl alcohol, about 1 to about 10% w/w polyoxypropylene stearyl ether and a hydrocarbon propellant system comprising one or more of propane, butane, and isobutane.

Yet another embodiment is a pharmaceutical foam composition comprising about 0.0643% w/w betamethasone dipropionate and about 0.0052% w/w calcipotriene with one or more pharmaceutically acceptable excipients, wherein the composition comprises about 86.9% w/w white petrolatum, about 3% w/w mineral oil, about 0.002% w/w alpha tocopherol, about 5% w/w isopropyl alcohol, about 5% w/w polyoxypropylene stearyl ether, and a hydrocarbon propellant system comprising one or more of propane, butane, and isobutane.

Yet another embodiment is a pharmaceutical foam composition comprising about 0.0643% w/w betamethasone dipropionate and about 0.0052% w/w calcipotriene with one or more pharmaceutically acceptable excipients, wherein the composition comprises about 86.9% w/w white petrolatum, about 3% w/w mineral oil, about 0.002% w/w alpha tocopherol, about 5% w/w isopropyl alcohol, about 5% w/w polyoxypropylene stearyl ether, and a hydrocarbon propellant system comprising one or more of propane, butane, and isobutane, wherein the composition has a viscosity of about 1 to about 15 poise.

Yet another embodiment is a pharmaceutical foam composition comprising about 0.0643% w/w betamethasone dipropionate and about 0.0052% w/w calcipotriene with one or more pharmaceutically acceptable excipients, wherein the composition comprises about 86.9% w/w white petrolatum, about 3% w/w mineral oil, about 0.002% w/w alpha tocopherol, about 5% w/w isopropyl alcohol, about 5% w/w polyoxypropylene stearyl ether, and a hydrocarbon propellant system comprising one or more of propane, butane, and isobutane, wherein the composition has related substance impurity C content of less than 3% after being stored at 30° C. and 65% relative humidity or 25° C. and 60% RH for 6 months.

Yet another embodiment is a method of treating psoriasis in a patient in need thereof by administering a foam composition of the present invention.

One embodiment is a method of treating psoriasis in a patient in need thereof by administering an effective amount of a pharmaceutical foam composition comprising about 0.0643% w/w betamethasone dipropionate and about 0.0052% w/w calcipotriene with one or more pharmaceutically acceptable excipients, wherein the composition comprises about 86.9% w/w white petrolatum, about 3% mineral oil, about 0.002% alpha tocopherol, about 5% isopropyl alcohol, about 5% polyoxypropylene stearyl ether, and a hydrocarbon propellant system comprising one or more of propane, butane, isobutane.

In another embodiment, the present invention relates to the use of pharmaceutical foam composition for the treatment of psoriasis in a patient in need thereof, wherein the composition comprises about 0.0643% w/w betamethasone dipropionate and about 0.0052% w/w calcipotriene with one or more pharmaceutically acceptable excipients comprising about 86.9% w/w white petrolatum, about 3% mineral oil, about 0.002% alpha tocopherol, about 5% isopropyl alcohol, about 5% polyoxypropylene stearyl ether and a hydrocarbon propellant system comprising one or more of propane, butane, and isobutane.

Yet another embodiment is a container system compatible for the storage of the foam composition of the present invention for topical administration to a patient in need thereof.

The container system may comprise an aerosol can and a valve assembly which further comprises an actuator, stem, gasket, mounting cup, housing and optionally a dip tube. In one preferred embodiment, the inner surface of the aerosol can is coated with an inert coating material. Suitable inert coating materials include, but are not limited to, an epoxy phenolic coating, microflex coating, modified polyester coating, acrylic coating and derivatives thereof.

In one embodiment, the container system comprises an aerosol can wherein the inner surface of the aerosol can is coated with an inert coating material.

In a further embodiment, the container system comprises an aerosol can wherein the inner surface of the aerosol can is coated with an inert coating material selected from epoxy phenolic coating, modified polyester coating, microflex coating, acrylic coating and derivatives thereof.

### DETAILED DESCRIPTION OF THE INVENTION

The terms used herein are defined as follows. If a definition set forth in the present application and a definition set forth later in a non-provisional application claiming priority from the present provisional application are in conflict, the definition in the non-provisional application shall control the meaning of the terms.

The term singular forms “a,” “an” and “the” include plural references unless the context clearly dictates otherwise. Thus, for example, reference to “an excipient” includes a single excipient as well as two or more different excipients, and the like.

Illustratively, the use of the term “about” with regard to a certain therapeutically effective pharmaceutical dose indicates that values slightly outside the cited values, e.g., plus or minus 0.1% to 10%, which are also effective and safe.

The term betamethasone dipropionate, unless otherwise indicates, refers to betamethasone dipropionate in free base or pharmaceutically acceptable salt forms, including hydrates, solvates, polymorphic forms, and complexes thereof.

The term calcipotriene refers to calcipotriene in free base or pharmaceutically acceptable salt forms, including hydrates, solvates, polymorphic forms, and complexes thereof.

The term “effective amount” or “therapeutically effective amount” denotes an amount of an active ingredient that, when administered to a subject for treating metabolic disorders, produces an intended therapeutic benefit in a subject.

The term “treating” or “treatment” as used herein includes the prophylaxis, mitigation, prevention, amelioration, or suppression of a disorder modulated by betamethasone propionate and calcipotriene in a mammal.

The term “patient” includes mammals, such as humans and domesticated animals, such as dogs and cats. Preferably, the patient is a human.

The term “pharmaceutically acceptable excipients” refers to the components of a pharmaceutical composition other than the active pharmaceutical ingredients and which are approved by regulatory authorities or are generally regarded as safe for human or animal use.

The term “combination” includes administration of one or more active pharmaceutical ingredients either in a single dosage form or in separate dosage forms, in fixed dose combination or administered separately as adjuvant therapy.

Throughout this specification it is to be understood that the words “comprise” and “include” and variations such as “comprises”, “comprising”, “includes”, “including” are to be interpreted inclusively, unless the context requires otherwise. That is, the use of these words may imply the inclusion of an element or elements not specifically recited.

Among the topical dosage forms, foam has gained high importance in the recent years and is emerging as an effective delivery system for topical delivery. It has numerous advantages over conventional dosage forms like creams, ointments, lotions. Foam dosage forms are patient compliant, spread easily on large skin surface areas, does not leave a greasy film after application and can be suitably used for drugs which cause irritation during application. The difficulty in conventional topical dosage forms is that patients are required to apply and rub it over the psoriatic lesion. This might be perceived as cumbersome and a dosage form such as a topical foam would provide a significant leap in patient compliance owing to the ease of application.

Foam is typically dispensed from a pressurized container containing a propellant. The foam container system comprises of an aerosol can and a valve assembly. The valve assembly further comprises an actuator, stem, gasket, mounting cup, housing and optionally a dip tube. For foam containers, another issue is the reaction of the active agents with the inner wall of the container.

Combination of betamethasone dipropionate and calcipotriene hydrate is marketed as an ointment under the trade name Taclonex®. U.S. Pat. No. 6,753,013 discloses an ointment dosage form of the two drugs.

An ointment dosage form for patient suffering from psoriatic lesion requires rubbing over the skin which is not patient compliant and hence a dosage form providing ease of application would be preferred. A foam composition is preferred over conventional topical dosage forms like ointment because foams are administered to a larger surface area which provides better absorption of the drug through the skin surface. Patient suffering from plaque psoriasis also face burning sensation and hence application of foam provide a cooling sensation at the site of administration which provides relief to the patient.

For the combination of betamethasone dipropionate and calcipotriene in foam dosage form, some of the key challenges are solubility of both the active ingredients, absorption of the active ingredient from the skin surface, viscosity of the formulation, dispensing of the foam from the container and maintaining stability of the formulation.

The pharmaceutical foam composition as per present invention comprises a betamethasone compound and calcipotriene suitable for topical administration. The composition may be topically administered to a patient in need thereof for the treatment of psoriasis, such as plaque psoriasis. The present invention also relates to a process for preparing the composition and filling it in the container system and its use in topical treatment of psoriasis. Preferably the invention relates to the topical administration of betamethasone dipropionate and calcipotriene with pharmaceutically acceptable excipients and propellants wherein the composition is stable in the container system coated with coating material selected from epoxy phenolic coatings, microflex coating, modified polyester coatings and acrylic coatings.

The corticosteroid compounds include, but not limited to betamethasone (9-fluoro-11,17,21-trihydroxy-16-methylpregna-1,4-diene-3,20-dione) and esters thereof such as the 21-acetate, 17-adamantoate, 17-benzoate, 17-valerate, and 17,21-dipropionate; alclomethasone and esters thereof such as the dipropionate; clobetasole and esters thereof such as the propionate; clobetasone and esters thereof such as the 17-butyrate; desoximetasone; diflucortolone and esters thereof, diflorasone and esters thereof such as the diacetate; fluocinonide; flumetasone and esters thereof such as the pivalate; fluocinolone and ethers and esters thereof such as the acetonide; fluticasone and esters thereof such as the propionate; fluprednidene and esters thereof such as the acetate; halcinonide; hydrocortisone and esters thereof such as the -17-butyrate; mometasone and esters thereof such as the furoate; and triamcinolone and ethers and esters thereof such as the acetonide. Preferably, the corticosteroid in the present invention is betamethasone dipropionate.

Betamethasone dipropionate is a synthetic corticosteroid bearing molecular weight of 504.6 and empirical formula C₂₈H₃₇FO₇.

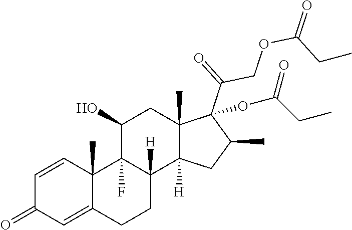

The term “Vitamin D analogue” is intended to indicate a synthetic compound comprising a vitamin D scaffold with side chain modifications and/or modifications of the scaffold itself. The analogue exhibits a biological activity on the vitamin D receptor comparable to that of naturally occurring vitamin D compounds. Vitamin D analogs include, but are not limited to calcipotriene, cholecalciferol, ergosterol, calcitriol, 25(OH)D3, dihydrotachysterol, 25-hydroxydihydrotachysterol, 1α-hydroxycholecalciferol, 25-hydroxycholecalciferol, calcipotriol, 22-oxacalcitriol, ergocalciferol, 1α-25-dihydroxyergocalciferol, 22,23-dihydroergocalciferol, 1,24,25-trihydroxycholecalciferol, previtamin D3, tacalciol, isovitamin D3, dihydrotachysterol3, (1S)-hydroxycalciol, (24R)-hydroxycalcidiol, 25-fluorocalciol, ercalcidiol, ertacalciol, (5E)-isocalciol, 22,23-dihydroercalciol, (24S)-methylcalciol, (5E)-(10S)-10,19-dihydroercalciol, (24S)-ethylcalciol, 22-dihydroergocalciferol, calcifidiol and (22E)-(24R)-ethyl-22,23-didehydrocalciol. Preferably, the Vitamin D analog is calcipotriene monohydrate. Calcipotriene is a synthetic Vitamin D3 analog bearing molecular weight of 430.6 and empirical formula C₂₇H₄₀O₃, H20.

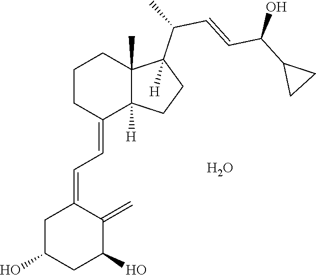

The term “Calcipotriene” as used herein, include acids, salts, esters, hydrates, polymorphs, hemihydrates, solvates, and derivatives thereof.

In an embodiment, the present invention relates to the stable pharmaceutical foam composition comprising betamethasone dipropionate and calcipotriene along with pharmaceutically acceptable excipients for the treatment of psoriasis.

The topical foam compositions of the present invention may include excipients comprising solvents, carriers, antioxidants, occlusive bases and a propellant.

The role of a solvent is to dissolve the pharmaceutically active agent. Non limiting examples of solvents selected from compounds of the general formula R³(OCH₂C(R¹)H)ₓOR² wherein x is in the range of 2-60, R¹ in each of the x units independently is H or CH3, R² is straight chain or branched C₁₋₂₀ alkyl or benzoyl, and R³ is H or phenylcarbonyloxy (polyoxypropylene-15-stearyl ether, polyoxyropylene-11-stearyl ether, polyoxypropylene-14-butyl ether, polyoxypropylene-10-cetyl ether or polyoxypropylene-3-myristyl ether), straight or branched C₂₋₄ alkyl esters of straight or branched C₁₀₋₁₈ alkanoic or alkenoic acids (Isopropyl myristate, isopropyl palmitate, isopropyl isostearate, isopropyl linolate or isopropyl monooleate), Propyleneglycol diesters with C₈₋₁₄ alkanoic acids (Propylene glycol dipelargonate), straight or branched C₈₋₂₄ alkanols or alkenol (Isopropyl, Capryl, pelargonic, capric, lauryl, cetyl, palmityl, stearyl, oleyl, linoelyl or myristyl alcohol or a branched C₈₋₂₄ alkanol (2-octyldodecanol), highly purified vegetable oils such as medium chain triglycerides or long chain triglycerides, N-alkylpyrrolidone or N-alkylpiperidone, Dodecyl 2-(N,N-dimethylamino) propionate (DDAIP). The topical foam composition of the present invention comprise about 0.1% w/w to about 5% w/w of a solvent, preferably isopropyl alcohol.

The pharmaceutically acceptable carrier used in the topical foam compositions of the present invention depends upon the solubility of the active agent and the nature of the composition. One of ordinary skill in the art would appreciate that given the wide range of pharmaceutically acceptable carriers known in the art, a suitable pharmaceutically acceptable carrier can be chosen to meet the requirements of the topical foam composition. A pharmaceutically acceptable carrier includes, but not limited to polyethylene glycols, glycerin, polyethylene glycols of various molecular weights and the like and mixtures thereof, propylene glycol, caffeine, xanthenes, isopropyl alcohol, propylene glycol, mineral oil, vegetable oil, silicon oil, lanolin, refined animal oil, isopropyl myristate, jojoba oil, capric-caprylic triglycerides of fractionated coconut oil, nutmeg oil, PEG-6 apricot kernel oil (Labrafil M 1944 CS), castor oil, olive oil and oleic acid, preferably mineral oil. The carrier used in the topical foam composition of the present invention is in an amount of about 1% w/w to about 10% w/w.

The topical foam composition may include at least one anti-oxidant. An effective amount of anti-oxidant can be added to the topical foam composition as described herein. The topical foam composition of the present invention comprise about 0.01% w/w to about 3% w/w of an anti-oxidant, preferably butylated hydroxytoluene.

The occlusive base of the present invention may comprise one or more ingredients, selected from the group comprising of, but not limited to, petrolatum or white petrolatum or paraffins, mineral oil, cetostearyl alcohol, cetyl alcohol, lanolin, stearic acid, crosslinked acrylic acid polymers such as the “carbomer” family of polymers, e.g., carboxypolyalkylenes that may be obtained commercially under the Carbopol® such as Carbopol®940, polyethylene oxides, polyoxyethylene-polyoxypropylene copolymers, polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin or mixtures thereof, preferably white petrolatum. The occlusive base used in the topical foam composition of the present invention is in an amount of about 40% w/w to about 95% w/w.

Further in an embodiment, the present invention relates to the pharmaceutical foam composition comprising betamethasone dipropionate 0.0643% and calcipotriene 0.0052% as active ingredients with one or more pharmaceutically acceptable excipients comprising white petrolatum 40-95%, mineral oil 1-10%, alpha tocopherol 0.001-0.01%, isopropyl alcohol 1-20%, polyoxypropylene stearyl ether 1-15% and propane, butane, iso butane as hydrocarbon propellant system.

Further in an embodiment, the present invention relates to the pharmaceutical foam composition comprising betamethasone dipropionate 0.0643% and calcipotriene 0.0052% as active ingredients with one or more pharmaceutically acceptable excipients comprising white petrolatum 86.9%, mineral oil 3%, alpha tocopherol 0.002%, isopropyl alcohol 5%, polyoxypropylene stearyl ether 5% and propane, butane, iso butane as hydrocarbon propellant system.

In an embodiment, the present invention relates to the method treating psoriasis in patients in need thereof by administering a pharmaceutical foam composition comprising betamethasone dipropionate 0.0643% and calcipotriene 0.0052% as active ingredients with one or more pharmaceutically acceptable excipients comprising white petrolatum 86.9%, mineral oil 3%, alpha tocopherol 0.002%, isopropyl alcohol 5%, polyoxypropylene stearyl ether 5% and propane, butane, iso butane as hydrocarbon propellant system.

In another embodiment, the present invention relates to the use of pharmaceutical foam composition for the treatment of psoriasis in patients in need thereof wherein the composition comprises betamethasone dipropionate 0.0643% and calcipotriene 0.0052% as active ingredients with one or more pharmaceutically acceptable excipients comprising white petrolatum 86.9%, mineral oil 3%, alpha tocopherol 0.002%, isopropyl alcohol 5%, polyoxypropylene stearyl ether 5% and propane, butane, iso butane as hydrocarbon propellant system.

In an embodiment, the present invention relates to an ointment based foam composition comprising betamethasone dipropionate 0.0643% and calcipotriene 0.0052% for topical administration in patients in need thereof for the treatment of psoriasis, wherein the foam breaks down after administration onto the skin and ointment forms an occlusive layer on affected area.

In another embodiment, the present invention relates to a stable foam composition wherein the composition is stable at 40° C./75% RH, 30° C./65% RH and 25° C./60% RH wherein the related substance impurity C content is not more than 3%.

In an embodiment, the present invention relates to the pharmaceutical foam composition comprising betamethasone dipropionate 0.0643% and calcipotriene 0.0052% as active ingredients with one or more pharmaceutically acceptable excipients comprising white petrolatum 86.9%, mineral oil 3%, alpha tocopherol 0.002%, isopropyl alcohol 5%, polyoxypropylene stearyl ether 5% and propane, butane, isobutane as hydrocarbon propellant system wherein, the composition has related substance impurity C content less than 3% when stored at 30° C./65% RH and 25° C./60% RH for 6 months

A stable composition is always desirable and becomes more critical in compositions containing more than one active agent. Further in case of foam compositions, it is ideal to check if there is any interaction between the active agents and the inner surface of the aerosol can. For this reason, the aerosol cans are coated with an inert material to protect the compositions from reacting with the inner surface of the aerosol can. The choice of the inert coating material becomes more critical in cases where the topical foam composition comprise a combination of two active agents viz. betamethasone dipropionate and calcipotriene.

In another embodiment, the present invention relates to the propellant system comprising hydrocarbon propellant or a mixture of hydrocarbon propellants.

The propellant provides the force that expels the product concentrate from the pressurized container in a particular form e.g. in the form of spray or foam or semisolid. When the composition is released from the pressurized container, the composition is topical foam. According to one embodiment, the topical foam is homogeneous. In another embodiment, the topical foam breaks easily with shear, such as gentle mechanical action e.g. rubbing or spreading. The propellants generally used are liquefied gas propellant and compressed gas propellant. The propellant generally used are hydrocarbons such propane or butane. The topical foam composition as per the present invention comprises a propellant selected from propane, butane, and isobutane and mixtures thereof. The topical foam compositions may include a hydrocarbon propellant or a mixture of hydrocarbon propellants. The propellant may be introduced into the composition at the time of filling, utilizing a pressurized container such as a standard dispenser. When the composition is released from the pressurized container, the composition is topical foam. The topical foam composition as per the present invention comprises about 20% w/w to about 60% w/w of a propellant, preferably n-propane, n-butane, isobutane or mixtures thereof.

Foam compositions are filled in the container system under high pressure. The mixture of active agents and excipients is filled into a container system and the propellant is subsequently filled under pressure. Propellant filling may be accomplished using cold fill process and the pressure fill process.

Cold fill process requires components which liquefy when cooled. This process requires cooling of both the foam composition and the aerosol can. The foam composition is added to the aerosol can followed by liquefied gas. This process is not suitable for aqueous solutions.

Pressure fill process is a convenient process and can be carried out at room temperature. This process requires filling of the foam composition into the aerosol can followed by inserting the valve assembly and crimping. The liquefied gas is then added under pressure through the valve. This process has a lesser danger of moisture contamination and propellant loss during the filling process is minimized. Preferably, the propellant of the present invention is filled into the container system by a pressure fill process.

The container for topical foam comprises an aerosol can and a valve assembly. The valve assembly comprises an actuator, stem, gasket, mounting cup, housing and optionally a dip tube. The container system also contains a propellant which is elemental in governing the dispensing properties of the foam composition. The container components also govern the characteristics of the topical foam composition such as the spray pattern, density, viscosity, and delivery parameters such as the particle size distribution and the delivery rate.

In another embodiment, the viscosity of the foam composition ranges between 1-15 poise.

In an embodiment, the Foam density of the present composition ranges from 0.2 to 0.5 g/ml

In an embodiment, the delivery rate of the foam composition dispensed from the container ranges between 0.5 to 1 g/sec

In another embodiment, the present invention relates to a container system compatible for the storage of the foam composition comprising betamethasone dipropionate and calcipotriene for topical administration to the patients in need thereof.

Further in an embodiment, the container system comprises an aerosol can and a valve assembly which further comprises an actuator, stem, gasket, mounting cup, housing and optionally a dip tube.

Aerosol cans are in direct contact with foam composition and hence is one of the most critical component of the container system. The aerosol cans are typically fabricated from metals such as tin plated steel, stainless steel, aluminium, glass or plastic. Glass containers are less susceptible to corrosion, offering better chemical compatibility and allow users to easily view the contents. Glass containers also allow for providing the maximum pressure safety and impact resistance and offer flexibility in design creativity. Typically glass containers can be used in products which have a low pressure and low percentage of propellants. Glass containers are not desirable in case of suspension formulations as they interfere with the aesthetics of the foam compositions. Tin plated steel provide necessary protection for most products. This material is light weight and cost effective. Stainless steel on the other hand is used in foam compositions where the container is required to be chemically resistant. Stainless steel container often is not preferred owing to the high cost. Plastic suffers from disadvantages because of the highly permeable nature and the high risk of interactions with the foam compositions.

Aluminium is widely used for foam compositions. Aluminium containers are easily fabricated by impact extrusion process which makes them seamless. Aluminium offers advantages as it is light in weight and less reactive as compared to other metal containers. Aluminium containers can be easily coated with coating materials to decrease interaction and are compatible with most coating materials. Aluminium containers are highly safe and pose fewer problems with respect to corrosion, leakage and incompatibility. The aerosol cans as per the present invention may be fabricated from aluminium and steel.

The aerosol cans of the container system as per the present invention are coated with an inert coating material. As per the present invention, the coating materials are selected from epoxy phenolic coatings, microflex coating, modified polyester coatings and acrylic coatings. It has been surprisingly found that the topical compositions of the present invention are compatible and stable with such coating materials. The coating layer may comprise of a single layer or multiple layer.

1. Epoxy phenolic coatings are made by reacting phenols with acetone or formaldehyde which are further reacted with epichlorhydrin. Diglycidyl ethers of what are called Bisphenol A epoxies, Bisphenol F epoxies, or phenolic novolac epoxies are the resultant materials. Crosslinking of resins cia polymerization reactions is done with various curing agents or blends of curing agents.

2. Polyester coating material is disclosed in U.S. Pat. No. 4,977,191 to Salsman, incorporated herein by reference. The Salsman '191 patent discloses an aqueous-based polyester resin. This is a reaction product of waste terephthalate polymer, at least one glycol and at least on oxyalkylated polyol. U.S. Pat. No. 5,281,630 to Salsman, describes the resin composition and the process of obtaining it. The Salsman '630 patent discloses an aqueous suspension of a sulfonated water-soluble or water dispersable polyester resin. This resin is a reaction product of terephthalate polymer, at least one glycol and at least one oxyalkylated polyol to produce a prepolymer resin. The prepolymer resin is further reacted with α,β-ethylenically unsaturated dicarboxylic acid. This resin is reacted with α,β-ethylenically unsaturated dicarboxylic acid residue to produce a sulfonated-terminated resin.

3. The acrylic coating material may include one or more non-functional monomers and one or more functional monomers (acid-functional monomers like acid-functional acrylic monomers). The acrylic component may include one or more vinyl monomers. The acrylic component is prepared through chain-growth polymerization using one or more ethylenically unsaturated monomers.

The combination and/or ratio(s) of the above monomers may be adjusted to provide a desired coating or film property.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1

| Sr.No. | Ingredients | % w/w |
|---|---|---|
| 1. | Calcipotriene Monohydrate IH | 0.0052 |
| 2. | Betamethasone Dipropionate USP | 0.0643 |
| 3. | Isopropyl alcohol USP | 5.0 |
| 4. | Mineral Oil USP | 3.0 |
| 5. | Vitamin E USP | 0.002 |
| | DL-Alpha Tocopherol | |
| 6. | White Petrolatum USP | 87.0 |
| 7 | Polyoxypropylene Stearyl Ether | 5.0 |

Propellants

| | | |
|---|---|---|
| 8. | Hydrocarbon Propellants | Qs |
| | (n-propane: n-butane: iso-butane) | |

### Manufacturing Process:

### A. Preparation of Oil Phase:

1. In stainless steel vessel, white petrolatum was added to quantity of mineral oil and quantity of polyoxypropylene stearyl ether
2. The mixture was heated at a temperature of 65-70° C. to form a clear liquid, ensuring complete melting of oil phase.

### B. Preparation of Drug Solution:

3. In another stainless steel vessel, polyoxypropylene stearyl ether and isopropyl alcohol was added and mixed under stirring
4. Calcipotriene Monohydrate was added in a mixture of Isopropyl alcohol and polyoxypropylene stearyl ether at continuous stirring to completely dissolve and form a clear solution.
5. Betamethasone dipropionate was then added to step 4 under stirring. Complete dissolution of Betamethasone dipropionate and Calcipotriene monohydrate was ensured
6. Drug phase was added to main manufacturing vessel containing oil phase
7. The mixture of step 6 was homogenized at 65-70° C. for 30-60 minutes under vacuum of 400-600 mm of HG. Then cooled down to Room Temperature.
8. Vitamin E solution was added to bulk of main manufacturing vessel under stirring
9. Required quantity of mixture of step 8 was filled in Aluminium containers. Aluminium containers were then crimped with valve cap.
10. Requisite amount of hydrocarbon propellant was filled in the container, after which the container was shaken for 10-15 minutes for complete dispersion of mixture in the hydrocarbon propellant.

### Example 2

Foam Density of the foam composition developed as per process and formulation mentioned in example 1 was calculated by following procedure

### Process:

1. The containers were maintained for 25° C. for at least 24 hours.
2. The container was shaken and 5 ml to 10 ml of foam was dispensed to waste.
3. A flat bottomed dish with a volume of about 60 ml and 35 mm high was tared
4. The actuator was pressed and the dish was filled uniformly using a circular motion.
5. The excess foam was leveled off with slide.
6. The mass of same volume of water by filling the same dish with water was determined
   Relative density is calculated as the ratio of m/e
   Where m=Mass of test sample of foam in grams
   e=mass of same volume of water in grams

### Example 3

The rate of delivery of amount of foam dispensed through the device was calculated by following procedure and is expressed as wt of foam dispensed per unit time.
1. Four aerosol containers were selected
2. If the label includes the directive, the containers were shaken.
3. After removing the caps and covers each valve was actuated for 2-3 sec.
4. Each container was weighed accurately (W1), and was immersed in a constant temperature bath until the internal pressure is equilibrated at a temperature of 25. The containers were removed from the bath and excess moisture was removed by blotting with a paper towel;
5. Each valve was actuated for 5.0 s (T) (accurately timed by use of a stopwatch); and each container was weighed again (W2).
6. The procedure was repeated three times for each container and average Delivery Rate, in g/s, for each container was calculated

Delivery Rate: (W1−W2)/T

W1: Initial weight of container before actuation.
W2: Weight of container after actuation for 5 seconds
T: Time in seconds.

### Example 4

Related substance impurity content was measured when samples were tested for stability at 40° C./75% RH, 30° C./65% RH and 25° C./60% RH up to 6 months.

Impurity C was found to be (5E, 7E, 22E, 24S)-24-cyclopropyl-9, 10-secochola-5,7,10 (19), 22 tetraene-1α,3β 24-triol 5E Isomer

The compositions developed as per example 1 were tested by both upright and inverted position of the device filled with foam composition

| | | | | 40° C./ | 30° C./ | 25° C./ |
|---|---|---|---|---|---|---|
| | | | | 75% | 65% | 60% |
| | | | | RH-3 | RH-3 | RH-3 |
| | | | | M- | M- | M - |
| Related Substances | | Limits | Initial | Upright | Upright | Upright |
| Calcipotriene | Impurity | NMT | Below | 0.34% | 0.55% | 0.22% |
| | C | 1.5% | LOQ | | | |

| | | | | 40° C./ | 30° C./ | 25° C./ |
|---|---|---|---|---|---|---|
| | | | | 75% | 65% | 60% |
| | | | | RH-3 | RH-3 | RH-3 |
| | | | | M- | M- | M- |
| Related Substances | | Limits | Initial | Inverted | Inverted | Inverted |
| Calcipotriene | Impurity | NMT | Below | 1.82% | 1.09% | 0.69% |
| | C | 1.5% | LOQ | | | |

1-10. (canceled)

11. A pharmaceutical foam composition comprising about 0.0643% w/w betamethasone dipropionate and about 0.0052% w/w calcipotriene with one or more pharmaceutically acceptable excipients, wherein
(i) the composition comprises about 40% w/w to about 95% w/w white petrolatum, about 1% w/w to about 10% w/w mineral oil, about 0.001% w/w to about 0.01% w/w alpha tocopherol, about 1% w/w to about 20% w/w isopropyl alcohol, about 1% w/w to about 10% w/w polyoxypropylene stearyl ether and a hydrocarbon propellant system comprising one or more of propane, butane, and isobutane,
(ii) the composition has a viscosity of 1 poise to 15 poise, and
(iii) the composition does not contain monomethyl ether or dimethyl ether or any derivative thereof.

12. The composition as claimed in claim 11, wherein the composition is stable in the container system coated with a coating material selected from epoxyphenol resin, modified polyesters, microflex coating or polyacrylates.

13. The composition as claimed in claim 12, wherein the container system comprise an aerosol can and a valve assembly, which further comprises an actuator, stem, gasket, mounting cup, housing and optionally a dip tube, wherein the inner surface of the aerosol can is coated with an inert coating material.

14. The composition as claimed in claim 13, wherein inert coating material is selected from an epoxy phenolic coating, microflex coating, modified polyester coating, acrylic coating and derivatives thereof.

15. The composition as claimed in claim 11, wherein after storage at 40° C. and 75% relative humidity for 3 months, the release substance impurity C ((5E,7E,22E,24S)-24-cyclopropyl 9,10-secochola-5,7,10(19),22-tetraene-1a,3b 24-triol 5E isomer) content is not more than 3%.

16. The composition as claimed in claim 11, wherein the composition has a density from 0.2 g/ml to 0.5 g/ml.

17. The composition as claimed in claim 12, wherein the delivery rate of the foam composition dispensed from the container ranges from 0.5 g/sec to 1 g/sec.
